(19) United States
(12) Patent Application Publication
(10) Pub. No.: US 2026/0078327 A1
(43) Pub. Date: Mar. 19, 2026

(54) DRUG TESTING PLATFORM FOR SIMULATING LUNG ENVIRONMENT

(71) Applicant: National Tsing Hua University, Hsinchu City (TW)

(72) Inventors: Yi-Ting KE, Hsinchu City (TW); Cheng-Hsien LIU, Hsinchu City (TW)

(21) Appl. No.: 18/912,166

(22) Filed: Oct. 10, 2024

(30) Foreign Application Priority Data
Sep. 19, 2024 (TW) 113135481

(51) Int. Cl. C12M 3/06; C12M 1/02; C12M 3/00

(52) U.S. Cl. CPC C12M 23/16; C12M 23/48; C12M 27/00

(57) ABSTRACT

A drug testing platform for simulating a lung environment includes a microfluidic chip and a stretching device. The microfluidic chip defines a microfluidic channel structure. The microfluidic channel structure includes a test unit, a cell shunt unit and a drug shunt unit. The stretching device is configured to stretch and contract the test unit in a manner of a reciprocal motion, thereby making degree of deformation of a plurality of test area of the test unit to be same during the reciprocal motion. The drug testing platform for simulating the lung environment according to the present disclosure may configure an environment that simulates a breathing motion of a human lung, and obtain multiple results within a single test.

### CROSS-REFERENCE TO RELATED APPLICATION

This application claims priority to Taiwanese Invention Patent Application No. 113135481, filed on Sep. 19, 2024, the entire disclosure of which is incorporated by reference herein.

### FIELD

The disclosure relates to a drug testing platform, and more particularly to a drug testing platform for testing a drug for treating lung cancer.

### BACKGROUND

One existing technique for testing and screening a drug for lung cancer treatment involves cell culture of lung cancer in a microfluidic chip which can simulate breathing motion of a human lung and injection of a drug for lung cancer therein, so as to obtain a test result of how the drug affects the lung cancer cell. This kind of microfluidic chip can only generate one test result at a time and cannot generate multiple test results simultaneously. As a result, multiple tests need to be conducted to thereby select the most suitable drug for lung cancer.

In clinical practice, multiple drugs for lung cancer usually need to be tested for screening out the most suitable drug for lung cancer. One can easily imagine that a considerable amount of time for multiple tests is required for screening out the most suitable drug for lung cancer when using this kind of microfluidic chip.

### SUMMARY

Therefore, an object of the disclosure is to provide a drug testing platform for simulating a lung environment that can configure an environment similar to breathing motion of a human lung and effectively screening the drug for lung cancer treatment.

Thus, a drug testing platform for simulating a lung environment according to the present disclosure includes a microfluidic chip and a stretching device.

The microfluidic chip defines a microfluidic channel structure. The microfluidic channel structure includes a test unit, a cell shunt unit and a drug shunt unit.

The test unit includes a first test area, a second test area, a third test area, a fourth test area, a fifth test area, a sixth test area, a seventh test area, an eighth test area, and a ninth test area which are spaced apart from each other and juxtaposed arranged. Each of the second test area, the fifth test area and the eighth test area has an upper part, a lower part, and a communication part that is disposed between the upper part and the lower part and that is formed with a plurality of vias.

The cell shunt unit includes a first cell shunt and a second cell shunt which are independent from each other. The first cell shunt is in fluid communication with the first test area, the upper part of the second test area, the fourth test area, the upper part of the fifth test area, the seventh test area and the upper part of the eighth test area. The second cell shunt is in fluid communication with the lower part of the second test area, the third test area, the lower part of the fifth test area, the sixth test area, the lower part of the eighth test area, and the ninth test area.

The drug shunt unit includes a first drug shunt, a second drug shunt, and a drug mixing channel which is in fluid communication with the first drug shunt and the second drug shunt. The first drug shunt is in fluid communication with the first test area, the second test area and the third test area. The second drug shunt is in fluid communication with the seventh test area, the eighth test area and the ninth test area. The drug mixing channel is in fluid communication with the fourth test area, the fifth test area and the sixth test area.

The stretching device is configured to stretch and contract the test unit in a manner of a reciprocal motion, thereby making degree of deformation of each of the first test area, the second test area, the third test area, the fourth test area, the fifth test area, the sixth test area, the seventh test area, the eighth test area, and the ninth test area to be same during the reciprocal motion.

### BRIEF DESCRIPTION OF THE DRAWINGS

Other features and advantages of the disclosure will become apparent in the embodiment(s) with reference to the accompanying drawings, in which:

FIG. 1 is a perspective view of an embodiment of a drug testing platform for simulating a lung environment according to the present disclosure;

FIG. 2 is a top view of a microfluidic chip and a holder structure of the embodiment;

FIG. 3 is a fragmentary schematic cross-sectional view taken along line III-III of FIG. 2;

FIG. 4 is a schematic view of a middle channel plate of the microfluidic chip;

FIG. 5 is a schematic view of a lower channel plate of the microfluidic chip;

FIG. 6 is a schematic view of an upper channel plate of the microfluidic chip;

FIG. 7 is a schematic view of a perforated film of the microfluidic chip; and

FIG. 8 is an exploded perspective view of the microfluidic chip and the holder structure.

### DETAILED DESCRIPTION

Before the disclosure is described in greater detail, it should be noted that similar components are represented by the same reference numerals in the following description.

The present disclosure will be further explained by the following embodiment(s). However, it should be understood that the embodiment(s) is(are) for illustrative purposes only and should not be interpreted as a limitation for the present disclosure.

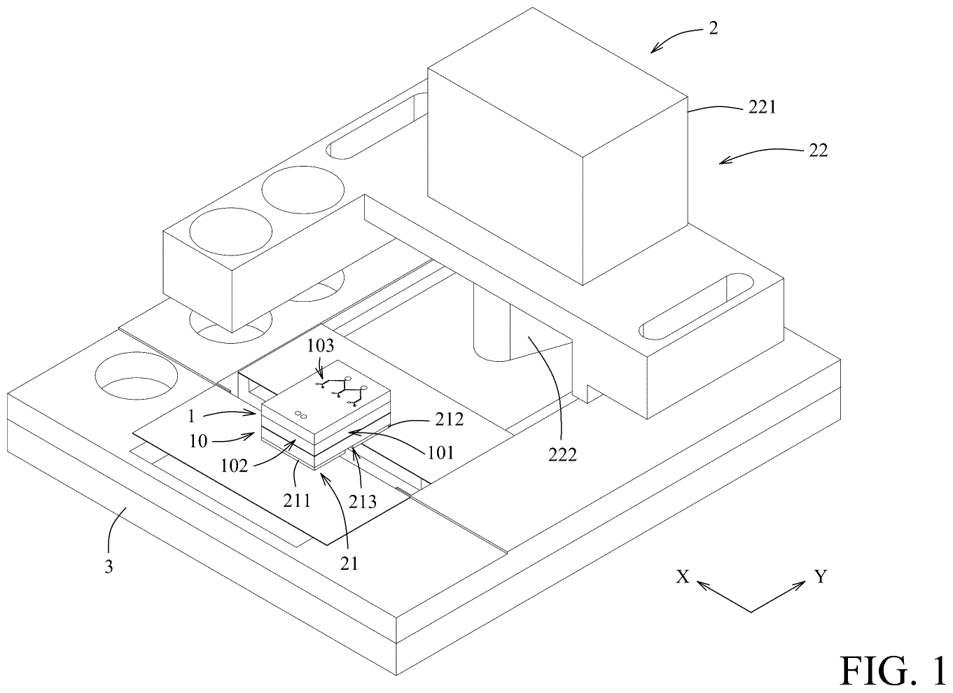

Referring to FIG. 1, an embodiment of a drug testing platform for simulating a lung environment is provided and includes a microfluidic chip 1 and a stretching device 2. The drug testing platform for simulating the lung environment in the present disclosure may configure an environment that simulates breathing motion of a human lung, and provides one or two drugs for treating lung cancer and allowing the drugs to react with one or two lung cancer cells in the environment. A usage of the drug testing platform for simulating the lung environment according to the present disclosure may obtain multiple test results with a single test.

The microfluidic chip 1 defines a microfluidic channel structure 10. The microfluidic channel structure 10 includes a test unit 101, a cell shunt unit 102 and a drug shunt unit 103.

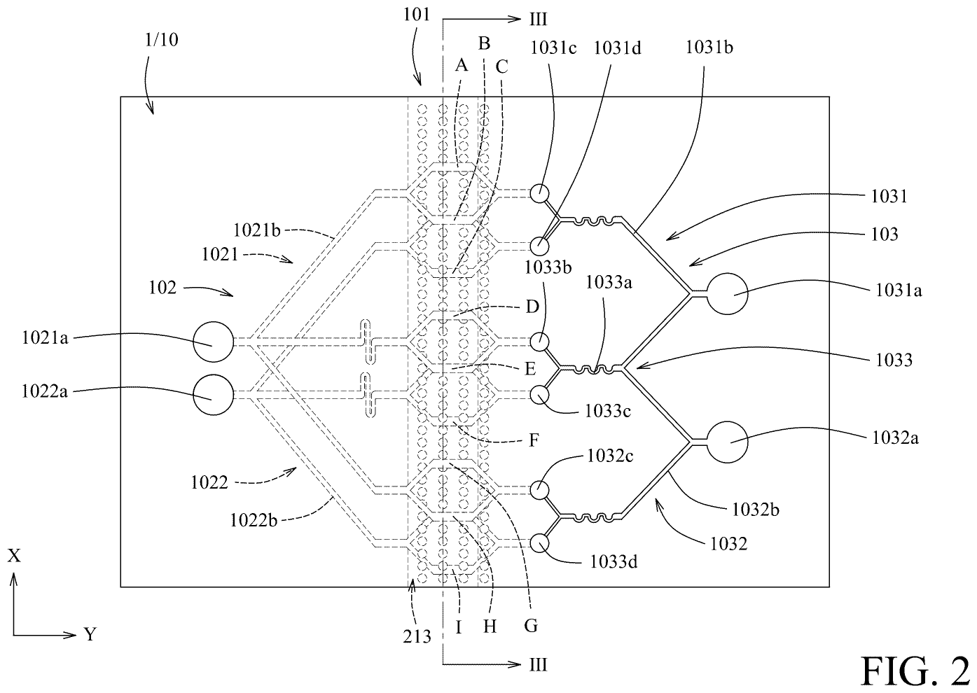

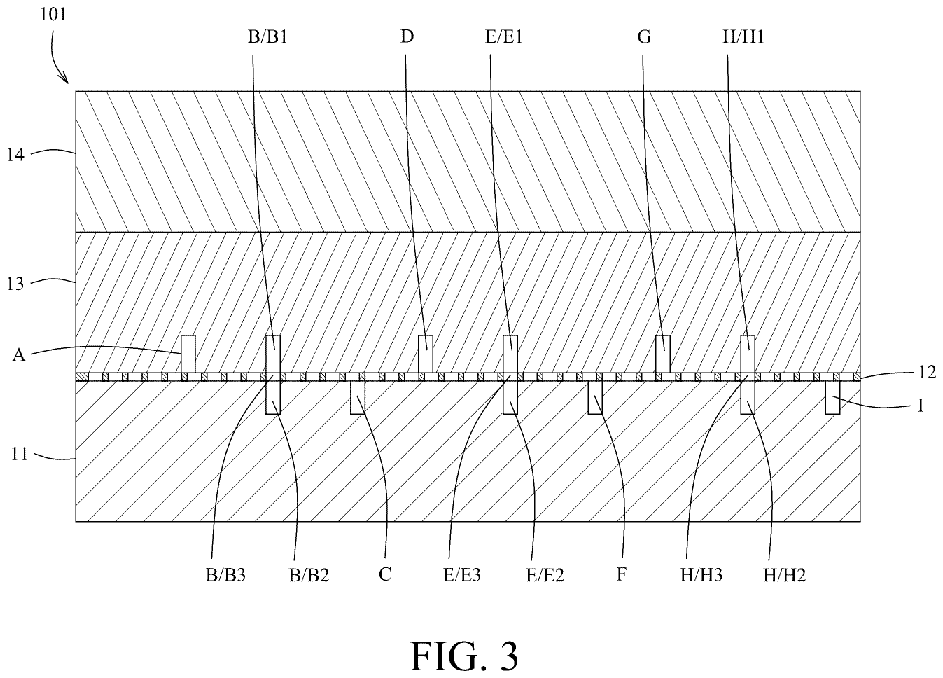

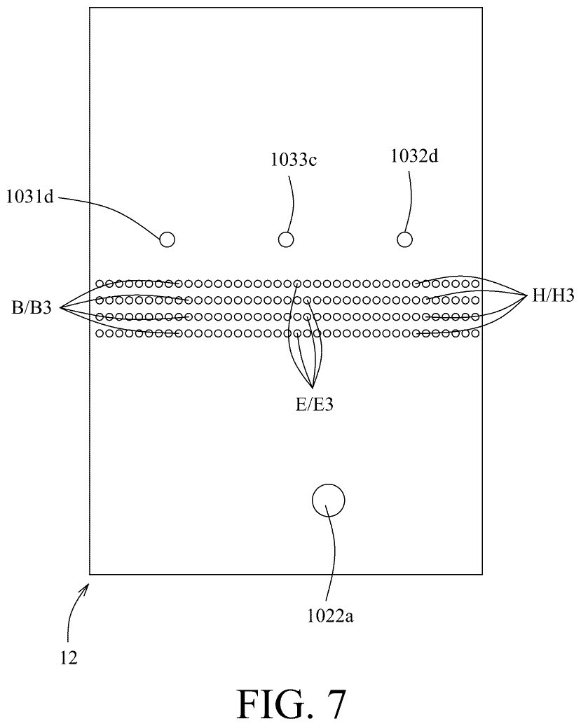

Referring to FIGS. 2 and 3, the test unit 101 that includes a first test area (A), a second test area (B), a third test area (C), a fourth test area (D), a fifth test area (E), a sixth test area (F), a seventh test area (G), an eighth test area (H), and a ninth test area (I) which are spaced apart from each other and juxtaposed arranged. The first test area (A), the second test area (B), the third test area (C), the fourth test area (D), the fifth test area (E), the sixth test area (F), the seventh test area (G), the eighth test area (H), and the ninth test area (I) are separate from each other in a first direction (X). Each of the first test area (A), the fourth test area (D) and the seventh test area (G) is used for accommodating a first cell hydrogel composition (for example, but not limiting to, a combination of a human lung adenocarcinoma cell A549 and a hydrogel). Each of the third test area (C), the sixth test area (F) and the ninth test area (I) is used for accommodating a second cell hydrogel composition (for example, but not limiting to, a combination of a human lung fibroblast Wi38 and a hydrogel). Each of the first test area (A), the third test area (C), the fourth area (D), the sixth test area (F), the seventh test area (G) and the ninth test area (I) is an independent chamber. The second test area (B) has an upper part (B1), a lower part (B2) and a communication part (B3) that is formed with a plurality of vias (see FIG. 7). Similar to the second test area (B), the fifth test area (E) has an upper part (E1), a lower part (E2) and a communication part (E3) that is formed with a plurality of vias (see FIG. 7). The eighth test area (H) has an upper part (H1), a lower part (H2) and a communication part (H3) that is formed with a plurality of vias (see FIG. 7). Since the second test area (B), the fifth area (E) and the eighth test area (H) are similar in configuration, the second test area (B) will be used as an example for further explanation below. The upper part (B1) is used for accommodation of the first cell hydrogel composition, and the lower part (B2) is used for accommodation of the second cell hydrogel composition. In order to make the upper part (B1) partially communicating with the lower part (B2), the communication part (B3) is disposed between the upper part (B1) and the lower part (B2).

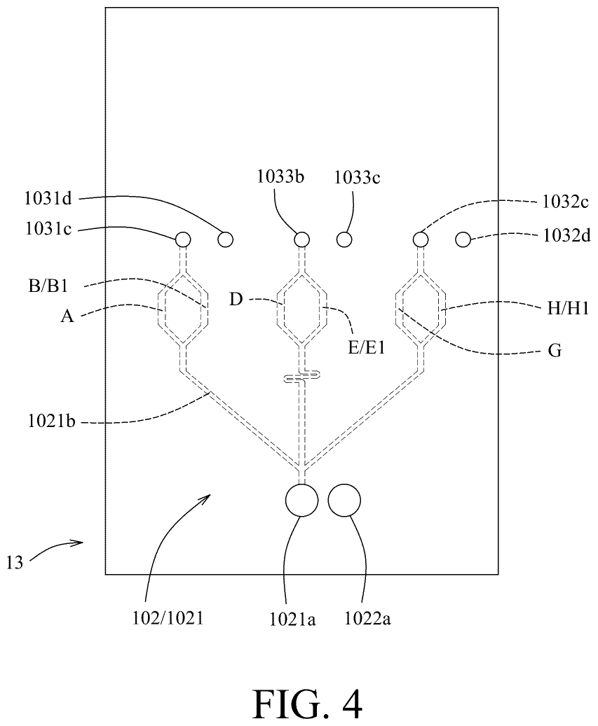

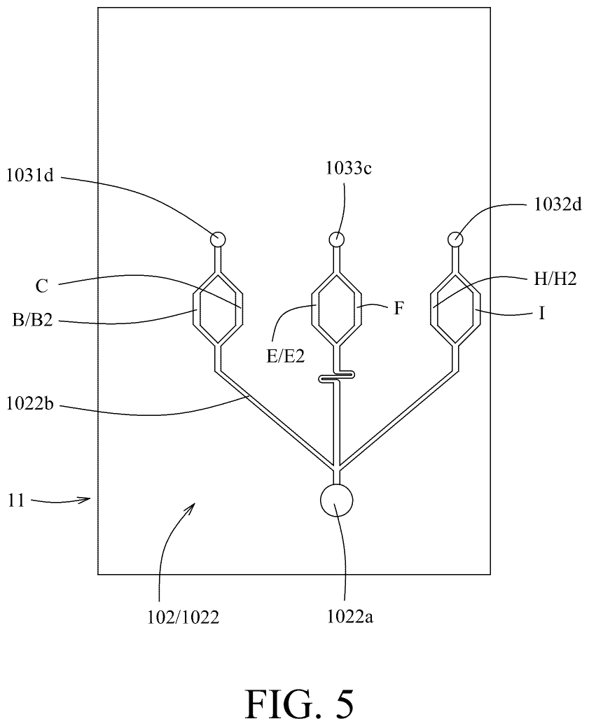

Referring to FIGS. 2, 4 and 5, the cell shunt unit 102 includes a first cell shunt 1021 and a second cell shunt 1022 which are independent from each other. Referring to FIGS. 2 and 4, the first cell shunt 1021 is in fluid communication with the first test area (A), the upper part (B1) of the second test area (B), the fourth test area (D), the upper part (E1) of the fifth test area (E), the seventh test area (G) and the upper part (H1) of the eighth test area (H) so as to divide the first cell hydrogel composition into branches and inject the first cell hydrogel composition into the first test area (A), the upper part (B1) of the second test area (B), the fourth test area (D), the upper part (E1) of the fifth test area (E), the seventh test area (G) and the upper part (H1) of the eighth test area (H). Referring to FIGS. 2 and 5, the second cell shunt 1022 is in fluid communication with the lower part (B2) of the second test area (B), the third test area (C), the lower part (E2) of the fifth test area (E), the sixth test area (F), the lower part (H2) of the eighth test area (H), and the ninth test area (I) so as to divide the second cell hydrogel composition into branches and inject the second cell hydrogel composition into the lower part (B2) of the second test area (B), the third test area (C), the lower part (E2) of the fifth test area (E), the sixth test area (F), the lower part (H2) of the eighth test area (H), and the ninth test area (I).

Referring to FIG. 2, the drug shunt unit 103 includes a first drug shunt 1031, a second drug shunt 1032, and a drug mixing channel 1033 which is in fluid communication with the first drug shunt 1031 and the second drug shunt 1032. The first drug shunt 1031 is in fluid communication with the first test area (A), the second test area (B) and the third test area (C) so as to divide a first drug (for example, but not limiting to, Pemetrexed) into branches and inject the first drug into the first test area (A), the second test area (B) and the third test area (C). The second drug shunt 1032 is in fluid communication with the seventh test area (G), the eighth test area (H) and the ninth test area (I) so as to divide a second drug (for example, but not limiting to, Cisplatin) into branches and inject the second drug into the seventh test area (G), the eighth test area (H) and the ninth test area (I). The drug mixing channel 1033 is in fluid communication with the fourth test area (D), the fifth test area (E) and the sixth test area (F) so as to mix the first drug and the second drug together, and divide them into branches, and inject them into the fourth test area (D), the fifth test area (E) and the sixth test area (F).

As a result, a test of effectiveness of the first drug on the first cell hydrogel composition is conducted in the first test area (A). A test of effectiveness of the first drug on the first cell hydrogel composition and on the second cell hydrogel composition is conducted simultaneously in the second test area (B). A test of effectiveness of the first drug on the second cell hydrogel composition is conducted in the third test area (C). A test of effectiveness of a mixture the first drug and the second drug (hereinafter referred to as the drug mixture) on the first cell hydrogel composition is conducted in the fourth test area (D). A test of effectiveness of the drug mixture on the first cell hydrogel composition and on the second cell hydrogel composition is conducted simultaneously in the fifth test area (E). A test of effectiveness of the drug mixture on the second cell hydrogel composition is conducted in the sixth test area (F). A test of effectiveness of the second drug on the first cell hydrogel composition is conducted in the seventh test area (G). A test of effectiveness of the second drug on the first cell hydrogel composition and on the second cell hydrogel composition is conducted simultaneously in the eighth test area (H). A test of effectiveness of the second drug on the second cell hydrogel composition is conducted in the ninth test area (I). That is say, a single test is conducted and generates a test result in a test area. Therefore, an overall of nine test results to be obtained.

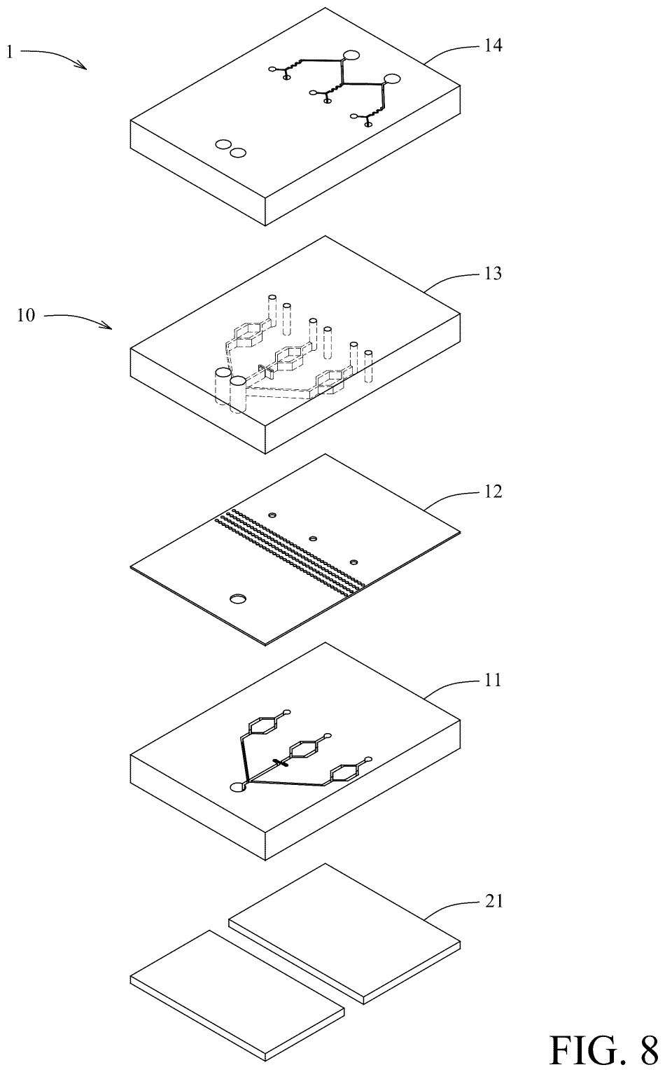

Specifically, referring to FIG. 2 in combination with FIG. 8, the microfluidic chip 1 includes a lower channel plate 11, a perforated film 12, a middle channel plate 13 and an upper channel plate 14 that are sequentially arranged in such order from bottom to top. The lower channel plate 11, the perforated film 12, the middle channel plate 13 and the upper channel plate 14 cooperate to define the microfluidic channel structure 10.

Firstly, the configuration of the test unit 101 in the lower channel plate 11, the perforated film 12 and the middle channel plate 13 are explained in detail. Referring to FIG. 4, the first test area (A), the upper part (B1) of the second test area (B), the fourth test area (D), the upper part (E1) of the fifth area (E), the seventh test area (G) and the upper part (H1) of the eighth area (H) are sequentially formed in the middle channel plate 13. Referring to FIG. 5, the lower part (B2) of the second test area (B), the third test area (C), the lower part (E2) of the fifth test area (E), the sixth test area (F), the lower part (H2) of the eighth area (H) and the ninth test area (I) are formed in the lower channel plate 11 in this order. Referring to FIG. 7 in combination with FIGS. 2 and 3, the perforated film 12 has a plurality of vias, in which some of the vias, serving as the communication part (B3) of the second test area (B), are used to fluidly communicate the upper part (B1) of the second test area (B) and the lower part (B2) of the second test area (B), some of the vias, serving as the communication part (E3) of the fifth test area (E), are used to fluidly communicate the upper part (E1) of the fifth test area (E) and the lower part (E2) of the fifth test area (E), and some of the vias, serving as the communication part (H3) of the eighth test area (H), are used to fluidly communicate the upper part (H1) of the eighth test area (H) and the lower part (H2) of the eighth test area (H). It should be noted that although the first test area (A), the third test area (C), the fourth test area (D), the sixth test area (F), the seventh test area (G) and the ninth test area (I) are in fluid communication with a portion of the vias, these test areas (A, C, D, F, G, I) are not in fluid communication with each other nor are in fluid communication with the test areas (B, E, H).

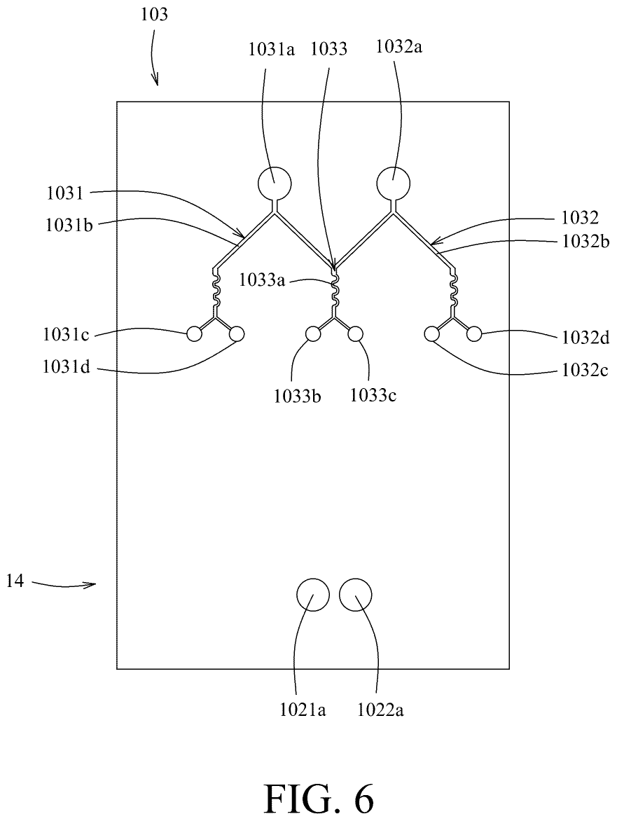

Next, the configuration of the cell shunt unit 102 in the lower channel plate 11, the perforated film 12 and the middle channel plate 13 and the upper channel plate 14 will be explained in detail. Referring to FIGS. 4 and 6, the first cell shunt 1021 includes a first cell injection hole 1021a and a first cell channel 1021b that is connected to the first cell injection hole 1021a. The first cell injection hole 1021a penetrates the upper channel plate 14 and extends to the middle channel plate 13. The first cell channel 1021b is formed in the middle channel plate 13 and extends from the first cell injection hole to be divided into three branches so as to be in fluid communication with the first test area (A), the upper part (B1) of the second test area (B), the fourth test area (D), the upper part (E1) of the fifth test area (E), the seventh test area (G) and the upper part (H1) of the eighth test area (H). In other words, the first cell hydrogel composition may be injected from the first cell injection hole 1021a and flow into the first cell channel 1021b, thereby flowing into the first test area (A), the upper part (B1) of the second test area (B), the fourth test area (D), the upper part (E1) of the fifth test area (E), the seventh test area (G) and the upper part (H1) of the eighth test area (H). Referring to FIGS. 4, 5, 6 and 7, the second cell shunt 1022 includes a second cell injection hole 1022a and a second cell channel 1022b that is connected to the second cell injection hole 1022a. The second cell injection hole 1022a sequentially penetrates the upper channel plate 14, the middle channel plate 13, and the perforated film 12 to extend to the lower channel plate 11. The second cell channel 1022b is formed in the lower channel plate 11 and extends from the second cell injection hole 1022a to be divided into three branches so as to be in fluid communication with the lower part (B2) of the second test area (B), the third test area (C), the lower part (E2) of the fifth test area (E), the sixth test area (F), the lower part (H2) of the eighth test area (H) and the ninth test area (I). In other words, the second cell hydrogel composition may be injected from the second cell injection hole 1022a and flow into the second cell channel 1022b, thereby flowing into the lower part (B2) of the second test area (B), the third test area (C), the lower part (E2) of the fifth test area (E), the sixth test area (F), the lower part (H2) of the eighth test area (H) and the ninth test area (I).

Then, the configuration of the drug shunt unit (103) in the lower channel plate (11), the perforated film (12), the middle channel plate (13) and the upper channel plate 14 will be explained in detail. Referring to FIGS. 4, 5, 6 and 7, the first drug shunt 1031 includes a first drug injection hole 1031a, a first communication channel 1031b, a first short channel 1031c and a first long channel 1031d. The first drug injection hole 1031a is formed on the upper channel plate 14. The first communication channel 1031b is formed on the upper channel plate 14 and is connected to the first drug injection hole 1031a. The first short channel 1031c and the first long channel 1031d extend and are branched off from the first communication channel 1031b. In other words, the first drug may be injected from the first drug injection hole 1031a and flow into the first communication channel 1031b, thereby flowing into the first short channel 1031c and the first long channel 1031d. The first short channel 1031c penetrates the upper channel plate 14 and extends to the middle channel plate 13 so as to be in fluid communication with the first test area (A) and the upper part (B1) of the second test area (B). As a result, the first drug, which flows to the first short channel 1031c, may further flow to the first test area (A) and the upper part (B1) of the second test area (B). The first long channel 1031d sequentially penetrates the upper channel plate 14, the middle channel plate 13, and the perforated film 12 to extend to the lower channel plate 11 so as to be in fluid communication with the lower part (B2) of the second test area (B) and the third test area (C). As a result, the first drug, which flows to the first long channel (1031d), may further flow to the lower part (B2) of the second test area (B) and the third test area (C). Referring to FIGS. 4, 5, 6 and 7, the second drug shunt 1032 includes a second drug injection hole 1032a, a second communication channel 1032b, a second short channel 1032c and a second long channel 1032d. The second drug injection hole 1032a is formed on the upper channel plate 14 and spaced apart from the first drug injection hole 1031a. The second communication channel 1032b is formed on the upper channel plate 14 and is connected to the second drug injection hole 1032a. The second short channel 1032c and the second long channel 1032d extend and are branched off from the second communication channel 1032b. In other words, the second drug may be injected from the second drug injection hole 1032a and flow into the second communication channel 1032b, thereby flowing into the second short channel 1032c and the second long channel 1032d. The second short channel 1032c penetrates the upper channel plate 14 and extends to the middle channel plate 13 so as to be in fluid communication with the seventh test area (G) and the upper part (H1) of the eighth test area (H). As a result, the second drug, which flows to the second short channel 1032c, may further flow into the seventh test area (G) and the upper part (H1) of the eighth test area (H). The second long channel 1032d sequentially penetrates the upper channel plate 14, the middle channel plate 13, and the perforated film 12 to extend to the lower channel plate 11 so as to be in fluid communication with the lower part (H2) of the eighth test area (H) and the ninth test area (I). As a result, the second drug, which flows to the second long channel 1032d, may further flow into the lower part (H2) of the eighth test area (H) and the ninth test area (I). Referring to FIGS. 4, 5, 6 and 7, the drug mixing channel 1033 includes a mixing portion 1033a, a short transportation portion 1033b and a long transportation portion 1033c. The mixing portion 1033a is formed on the upper channel plate 14 and is connected to the first drug injection hole 1031a and the second drug injection hole 1032a. The short transportation portion 1033b and the long transportation portion 1033c extend and are branched off from the mixing portion 1033a. In other words, after the first drug from the first communication channel 1031b and the second drug from the second communication channel 1032b are mixed in the mixing portion 1033a and form the drug mixture, the drug mixture then flows to the short transportation portion 1033b and the long transportation portion 1033c. The short transportation portion 1033b penetrates the upper channel plate 14 and extends to the middle channel plate 13 so as to be in fluid communication with the fourth test area (D) and the upper part (E1) of the fifth test area (E). As a result, the drug mixture, which flows to the short transportation portion 1033b, may further flow to the fourth test area (D) and the upper part (E1) of the fifth test area (E). The long transportation portion 1033c sequentially penetrates the upper channel plate 14, the middle channel plate 13, and the perforated film 12 and extends to the lower channel plate 11 so as to be in fluid communication with the lower part (E2) of the fifth test area (E) and the sixth test area (F). As a result, the drug mixture, which flows to the long transportation portion 1033c, may further flow to the lower part (E2) of the fifth test area (E) and the sixth test area (F).

Referring to FIG. 1, a stretching device 2 includes a holder structure 21 that is used for holding the microfluidic chip 1, and a driving unit 22 that is used for driving the microfluidic chip 1 to stretch. In an embodiment of the present disclosure, the stretching device 2 is disposed on a substrate 3. The holder structure 21 includes a fixed portion 211, a movable portion 212 spaced apart from the fixed portion 211, and a groove 213 formed between the fixed portion 211 and the movable portion 212. The fixed portion 211 is mounted on the substrate 3 in an unmovable manner, while the movable portion 212 is disposed on the substrate 3 in a movable manner with respect to the fixed portion 211. The fixed portion 211 and the movable portion 212 respectively and fixedly hold two ends of the microfluidic chip 1 which are opposite to each other such that a normal projection of the test unit 101 of the microfluidic chip 1 on the holder structure 21 falls within the groove 213 completely. The driving unit 22 is connected to the movable portion 212 and adapted for driving the movable portion 212 to move reciprocally with respect to the fixed portion 211 so as to stretch and contract the test unit 101 reciprocally, thereby causing a deformation of the first test area (A) to the ninth test area (I) in the test unit 101. The deformation (that is to say, stretching and contracting) of the first test area (A) to the ninth test area (I) in the test unit 101 is used for simulating the environment of the human lung during the breathing motion, and is to make the first cell hydrogel composition and the second cell hydrogel composition which are accommodated therein be driven to deform, so that the lung cancer cell to be tested receives a mechanical force similar to the breathing motion of a human being. In order to facilitate the deformation of the first test area (A) to the ninth test area (I), the lower channel plate 11, the perforated film 12, the middle channel plate 13, and the upper channel plate 14 are made of a deformable material. The driving unit 22 may be any mechanism that can drive the movable portion 212 to move reciprocally with respect to the fixed portion 211. For example, the driving unit 22 has a driving module 221 and a cam 222. The driving module 221 may be controlled for driving the cam 222 so as to drive the movable portion 212 to move reciprocally with respect to the fixed portion 211. A specific configuration of the driving module 221 may include, but not limiting to, a servomotor (not depicted in the figures), and a controller that is used for controlling the servomotor (not depicted in the figures). The controller may be, for example, an Arduino Nano™ microprocessor. The manner of operation regarding the stretching devices 2 is disclosed in U.S. Patent No. US20230039490A1.

In addition, since the normal projection of the test unit 101 of the microfluidic chip 1 on the holder structure 21 falls within the groove 213 completely, a degree of deformation of the first test area (A) to the ninth test area (I) is similar without a significant difference. That is to say, each of the test areas (i.e., from the first test area (A) to the ninth test area (I)) is simulated to be within a similar environment, thereby ensuring that no additional factors are produced due to a difference in the degree of deformation in the first test area (A) to the ninth test area (I), which is conducive to accuracy and reliability of the test results. In other words, when the stretching device 2 operates, the stretching device 2 stretches and contracts the test unit 101 in a manner of a reciprocal motion, thereby making each of the first test area (A), the second test area (B), the third test area (C), the fourth test area (D), the fifth test area (E), the sixth test area (F), the seventh test area (G), the eighth test area (H), the ninth test area (I) to deform in the same degree during the reciprocal motion and to deform in a second direction (Y). The first direction (X) may be transverse to and/or perpendicular to the second direction (Y). In another embodiment (not depicted in the figures), the microfluidic chip 1 of the drug testing platform for simulating the lung environment in the present disclosure may include a plurality of microfluidic channel structures 10 which are juxtaposed arranged. In addition, a degree of deformation for the test unit 101 of each of the microfluidic channel structures 10 is the same in a single test. In yet another embodiment (not depicted in the figures), the drug testing platform for simulating the lung environment in the present disclosure may include a plurality of microfluidic chips 1. In addition, a degree of deformation for the test unit 101 of the microfluidic channel structure 10 of each of the microfluidic chips 1 is the same in a single test.

In summary, the drug testing platform for simulating the lung environment according to the present disclosure may simulate an environment of breathing motion of a human lung using the microfluidic chip 1 in cooperation with the stretching device 2. In such environment, one or two drugs for lung cancer may interact with one or two lung cancer cells. Moreover, the drug testing platform for simulating the lung environment may obtain nine precise results within a single test, so the purpose of the present disclosure is indeed achieved.

The above descriptions are merely embodiment(s) for the present disclosure and should not limit the scope of the implementation of this disclosure. Any simple equivalent changes and modifications made in accordance with the claims and descriptions of this disclosure are still within the claimed scope covered by this disclosure.

What is claimed is:

1. A drug testing platform for simulating a lung environment, comprising:
a microfluidic chip defining a microfluidic channel structure, said microfluidic channel structure including:
a test unit that includes a first test area, a second test area, a third test area, a fourth test area, a fifth test area, a sixth test area, a seventh test area, an eighth test area, and a ninth test area which are spaced apart from each other and juxtaposed arranged, wherein each of said second test area, said fifth test area and said eighth test area has an upper part, a lower part, and a communication part that is disposed between said upper part and said lower part and that is formed with a plurality of vias,
a cell shunt unit that includes a first cell shunt and a second cell shunt which are independent from each other, said first cell shunt being in fluid communication with said first test area, said upper part of said second test area, said fourth test area, said upper part of said fifth test area, said seventh test area and said upper part of said eighth test area, said second cell shunt being in fluid communication with said lower part of said second test area, said third test area, said lower part of said fifth test area, said sixth test area, said lower part of said eighth test area, and said ninth test area, and
a drug shunt unit that includes a first drug shunt, a second drug shunt, and a drug mixing channel which is in fluid communication with said first drug shunt and said second drug shunt, said first drug shunt being in fluid communication with said first test area, said second test area and said third test area, said second drug shunt being in fluid communication with said seventh test area, said eighth test area and said ninth test area, said drug mixing channel being in fluid communication with said fourth test area, said fifth test area and said sixth test area; and
a stretching device configured to stretch and contract said test unit in a manner of a reciprocal motion, thereby making degree of deformation of each of said first test area, said second test area, said third test area, said fourth test area, said fifth test area, said sixth test area, said seventh test area, said eighth test area, and said ninth test area to be same during the reciprocal motion.

2. The drug testing platform for simulating the lung environment as claimed in claim 1, wherein said microfluidic chip includes a lower channel plate, a perforated film, a middle channel plate and an upper channel plate that are sequentially arranged in such order from bottom to top, and said lower channel plate, said perforated film, said middle channel plate and said upper channel plate cooperate to define said microfluidic channel structure.

3. The drug testing platform for simulating the lung environment as claimed in claim 2, wherein said first test area, said upper part of said second test area, said fourth test area, said upper part of said fifth area, said seventh test area and said upper part of said eighth area are sequentially formed in said middle channel plate, said lower part of said second test area, said third test area, said lower part f said fifth test area, said sixth test area and said lower part of said eighth area and said ninth test area being sequentially formed in said lower channel plate, said communication part of said second test area, said communication part of said fifth test area, and said communication part of said eighth test area being formed in said perforated film.

4. The drug testing platform for simulating the lung environment as claimed in claim 3, wherein said first cell shunt includes a first cell injection hole and a first cell channel that is connected to said first cell injection hole, said first cell injection hole penetrating said upper channel plate and extending to said middle channel plate, said first cell channel being formed in said middle channel plate and extending from said first cell injection hole to be divided into three branches so as to be in fluid communication with said first test area, said upper part of said second test area, said fourth test area, said upper part of said fifth test area, said seventh test area and said upper part of said eighth test area.

5. The drug testing platform for simulating the lung environment as claimed in claim 3, wherein said second cell shunt includes a second cell injection hole and a second cell channel that is connected to said second cell injection hole, said second cell injection hole sequentially penetrating said upper channel plate, said middle channel plate, said perforated film to extend to said lower channel plate, said second cell channel being formed in said lower channel plate and extending from said second cell injection hole to be divided into three branches so as to be in fluid communication with said lower part of said second test area, said third test area, said lower part of said fifth test area, said sixth test area, said lower part of eighth test area and said ninth test area.

6. The drug testing platform for simulating the lung environment as claimed in claim 3, wherein said first drug shunt includes a first drug injection hole, a first communication channel, a first short channel and a first long channel, said first drug injection hole being formed on said upper channel plate, said first communication channel being formed on said upper channel plate and connected to said first drug shunt, said first short channel and said first long channel extending and being branched off from said first communication channel, said first short channel penetrating said upper channel plate and extending to said middle channel plate so as to be in fluid communication with said first test area and said upper part of said second test area, said first long channel sequentially penetrating said upper channel plate, said middle channel plate, and said perforated film to extend to said lower channel plate so as to be in fluid communication with said lower part of said second test area and said third test area.

7. The drug testing platform for simulating the lung environment as claimed claim 6, wherein said second drug shunt includes a second drug injection hole, a second communication channel, a second short channel and a second long channel, said second drug injection hole being formed on said upper channel plate and spaced apart from said first drug injection hole, said second communication channel being formed on said upper channel plate and connected to said second drug injection hole, said second short channel and said second long channel extending and being branched off from said second communication channel, said second short channel penetrating said upper channel plate and extending to said middle channel plate so as to be in fluid communication with said seventh test area, said upper part of said eighth test area, said second long channel sequentially penetrating said upper channel plate, said middle channel plate, and said perforated film to extend to said lower channel plate so as to be in fluid communication with said lower part of said eighth test area and said ninth test area.

8. The drug testing platform for simulating the lung environment as claimed in claim 7, wherein said drug mixing channel includes a mixing portion, a short transportation portion and a long transportation portion, said mixing portion being formed on said upper channel plate and connected to said first drug injection hole and said second drug injection hole, said short transportation portion and said long transportation portion extending and branched off from said mixing portion, said short transportation portion penetrating said upper channel plate and extending to said middle channel plate so as to be in fluid communication with said fourth test area and said upper part of said fifth test area, said long transportation portion sequentially penetrating said upper channel plate, said middle channel plate, and said perforated film and extending to said lower channel plate so as to be in fluid communication with said lower part of said fifth test area and said sixth test area.

9. The drug testing platform for simulating the lung environment as claimed in claim 1, wherein said stretching device includes:
a holder structure that includes a fixed portion, a movable portion spaced apart from said fixed portion and a groove formed between said fixed portion and said movable portion, said fixed portion and said movable portion respectively holding fixedly two ends of said microfluidic chip which are opposite to each other such that a normal projection of said test unit of said microfluidic chip on said holder structure falls within said groove completely, and
a driving unit that is connected to said movable portion and adapted for driving said movable portion to move reciprocally with respect to said fixed portion so as to stretch and contract said test unit in the manner of the reciprocal motion.

10. The drug testing platform for simulating the lung environment as claimed in claim 1, wherein each of said first test area, said third test area, said first fourth area, said sixth test area, said seventh test area and said ninth test area is an independent chamber.

11. The drug testing platform for simulating the lung environment as claimed in claim 1, wherein:
said first test area, said second test area, said third test area, said fourth test area, said fifth test area, said sixth test area, said seventh test area, said eighth test area, and said ninth test area are separate from each other in a first direction; and
said first test area, said second test area, said third test area, said fourth test area, said fifth test area, said sixth test area, said seventh test area, said eighth test area, and said ninth test area deform in a second direction, the first direction being transverse to the second direction.
